(19) United States
(12) Patent Application Publication
(10) Pub. No.: US 2005/0143330 A1
(43) Pub. Date: Jun. 30, 2005

(54) Method for the treatment of Parkinson's Disease

(76) Inventors: Ron Mandel, Moraga, CA (US); Stuart Leff, Oakland, CA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/259,459

(22) Filed: Sep. 30, 2002

(51) Int. Cl. A61K 48/00

(52) U.S. Cl. 514/44; 424/93.2

(57) ABSTRACT

A method of treating Parkinson's Disease in patients exhibiting increasing resistance to the administration of L-dopa due to loss of aromatic L-amino acid decarboxylic activity in striatal neurons comprises transfection of the caudate and/or putamen regions with a viral vector encoding AADC. The vector preferably has a promoter system provided for the expression of the AADC nucleic acid, and is injected at a slow rate, at a level designed to restore AADC activity to tissues undergoing progressive loss of that activity. The AADC renewed activity permits conversion of L-dopa, in the brain, to dopamine.

### FIELD OF THE INVENTION

This invention pertains to a method of treating Parkinson's Disease. More specifically, a specific method for the introduction of viral vectors that can restore aromatic L-amino decarboxylase (AADC) activity to striatal neurons which have lost this activity during the progression of the disease is provided. Restoration of the activity, coupled with systemic administration of the dopamine pro-drug, L-dopa, provides a method for treatment, given provision of an appropriate delivery protocol.

### BACKGROUND OF THE INVENTION

Among neurodegenerative diseases, Parkinson's Disease provides a good example of a localized disorder characterized by slow degeneration. Specifically, degeneration of dopamine-producing neurons, located in the striatum, and in particular, the caudate or putamen causes a loss of up to eighty percent (80%) of the dopamine, which in healthy individuals interacts with post-synaptic dopaminergic receptors. Replacement of lost dopamine is complicated, because it cannot pass the blood-brain barrier of the central nervous system (CNS). An alternative strategy is to provide a precursor, L-dopa, which can pass the blood-brain barrier, and can be administered orally, conventionally several times a day. While frequently, patients initially respond quite well to L-dopa, as the disease progresses, AADC levels decrease, preventing adequate conversion into dopamine, and rendering the patient relatively insensitive to increased dosages, with reduced therapeutic benefits and increased side effects associated therewith. Mouradian et al., Ann. Neurol. 22, 475-479 (1987) and Neff, et al., Prog. Brain Res. 106, 91-97 (1995).

As an alternative, increasing interest has been focused on the delivery of heterologous genes for the expression of dopamine using viral vectors. U.S. Pat. No. 6,180,613, Kaplitt, et al., describe an adeno-associated virus (AAV) vector designed for the delivery of heterologous DNA to the central nervous system. The patent describes an alternative strategy to the treatment of Parkinson's Disease, by delivering a plurality of vectors used to transduce neurons in vivo. The disclosure of this patent is incorporated by reference. The patent focuses on the restoration of the entire dopamine pathway in the CNS.

In 1998, the inventors herein and others herein reported the administration of replication deficient adeno-associated viruses bearing either genes for the expression of AADC accompanied by a promoter system, or control administration of an adeno-associated virus carrying Lac Z with the CMV promoter, to the striatum of rats. The rats were 6-hydroxydopamine lesion rats. Surprisingly, both the control (Lac Z) and target (hAADC) groups showed the same dose-response for L-dopa-induced rotational behavior. The AADC treated rats showed an increase in striatal DA over a relatively short period of time. This successful transduction was demonstrated over a longer period of time of six months.

Similar attempts to demonstrate increased dopamine production are detailed in U.S. Pat. No. 6,103,226, Kang et al.. This patent focuses on an ex-vivo system of grafting cells onto the CNS, which cells have been previously treated by transfection with heterologous DNA that encodes the expression of tyrosine hydroxylase and GTP cyclohydrolase, enzymes upstream of AADC in the dopamine expression pathway.

Subsequent to Applicants' invention, additional reports of the utility of gene transfer of AADC to Parkinson models have been reported in the literature. Muramatsu, et al., Human Gene Therapy, 13, 345-354 (2002) again describes a multiple enzyme delivery, notes the prior work of Mandel and others, but concludes that there is no study confirming the suitability of AAV transduction in the treatment of Parkinson's Disease in humans and other primates. Sancheaz-Pernaute et al., Molecular Therapy, 4, 324-330 (October 2001) and U.S. Pat. No. 6,309,634 describe a different method for delivery of AAV vectors to the central nervous system, for the treatment of Parkinson's Disease. These references focus on the delivery of the rAAV virions carrying the heterologous genes for the expression of dopamine-related enzymes using convection-enhanced delivery, or CED. CED is characterized as broad delivery with a pump, such as an osmotic pump. These references specifically indicate rAAV injection to be undesirable, and call for CED to achieve broad delivery of the replication-defective virions.

Regrettably, none of the reported methods have been translated into effective treatments for Parkinson's Disease. This is in part due to a focus on rodent models. While the utility of the claimed invention, in terms of the effectiveness of injection, as opposed to CED, is discussed below in the context of rodents, these are not sufficient to establish an effective method for the treatment of Parkinson's Disease in humans. Among other problems presented, the rodent nigrostratal lesion Parkinson's Disease model is inadequate in it's behavioral deficient manifestation, because rats do not have a corticospinal system which allows for the adequate study of fine visual-motor movement. The anatomical organization of the basal ganglia also differs from humans and other primates, as opposed to rodents. Only primates, as opposed to rodents, have a discreet caudate and putamen, and topographical apposition of the internal and external segments of the globus pallidus. Thus, while rodent studies may be relied on to demonstrate the efficacy of rAAV transfection, that is, the ability to deliver heterologous DNA to the CNS and express the same, they are inadequate to predict the efficiency or effectiveness of treatment in humans.

Convection enhanced delivery, or CED, has also posed problems as a delivery method. By design, this method does not control delivery of the active agent, in this case, the virion and the AADC gene, to any particular region, and results in spread beyond the target cells which have lost dopaminergic activity. This results in a loss of therapeutic efficacy, and requires a much larger dosage, and CNS trauma, creating potential safety issues.

Accordingly, the art continues to search for a method of effectively treating Parkinson's Disease by restoration of dopaminergic activity to the CNS in humans.

### SUMMARY OF THE INVENTION

Applicants have discovered that direct injection of recombinant adeno-associated virus virions, which comprise a nucleic acid sequence encoding AADC, into those portions of the patient's brain striatum that have suffered loss of dopaminergic activity, particularly the caudate and putamen striatus, effectively restores the ability of the depleted cells to convert the pro-drug L-dopa, systemically administered, to dopamine, to ameliorate the course and symptoms of Parkinson's Disease.

The direct injection protocol of administration will be 1-2 injections for each site (caudate and putamen) which each dose being 2-10 μl, administered at a slow infusion rate of 0.05-0.5 μl/minute. Titre of the AAV-MD-hAADC replication-deficient verion is 1×10e12 particle/ml. Concentration ranges of 10e10-10e13 are contemplated.

### DETAILED DESCRIPTION OF THE INVENTION

The efficacy of this invention was first demonstrated using rodent models, that is, unilaterally 6-hydroxydopamine lesioned rats. The subjects received intrastriatal injections of either rAAV-CMV-LacZ or the active composition of rAAV-MD-hAADC on the lesioned side. Microdiayalis experiments were undertaken in the treated striatum, after stabilization, and after systemic administration of L-dopa. Both groups treated in this experiment showed the same dose-response for L-dopa-induced rotational behavior. The treated rat showed a striking increase in AADC activity to those receiving the control virus. Striatal AADC activity in the Lac Z group was shown to be 22% of normal, whereas rAAV-MD-HAADC transduction restored AADC activity to 66% of the striatum. Considered over a longer term of six months, similar control groups receiving rAAV-MD-hTH (tyrosine hydroxylase—another dopamine pathway enzyme) exhibited AADC activity reduced to less than 15% of their intact side. The HAADC treated rats exhibited AADC activity of 75% of the intact side. On systemic administration of L-dopa (which may be in the conventional L-dopa/carbidopa combination, or L-dopa alone).

This invention focuses on the treatment of Parkinson's Disease. Although other neurodegenerative diseases may be susceptible of treatment in a similar manner, given the lack of predictability in transferring results effective in one family of organisms, and one disease, to another related only in morphology, this invention is confined to the treatment of Parkinson's Disease. Although other primates characterized by Parkinson's Disease or conditions imitating Parkinson's Disease may be treated by the methods described and claimed below, this invention is directed specifically to the treatment of humans. The specific administration protocol was designed for upper primates, and humans, specifically.

The recombinant, replication-defective adeno-associated virus vector, together with the nucleic acid sequence for AADC, is widely available prior to the date of invention. The vector described in U.S. Pat. No. 6,180,613, for AADC can be employed.

Generally, an AAV vector is a vector derived from an adeno-associated virus stereotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, e.g., Kotin, R. M. (1994) Human Gene Therapy 5:793-801; Bems, K. I. “Parvoviridae and their Replication” in Fundamental Virology, 2nd Edition, (B. N. Fields and D. M. Knipe, eds.) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV stereotypes, including without limitation, AAV-1, AAV-2, AAV-3, AAV-4, AAV-5, AAVX7, etc. Furthermore, 5′ and 3′ ITRs which flank a selected nucleotide sequence in an AAV vector need not necessarily be identical or derived from the same AAV stereotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

Additionally, AAV ITRs may be derived from any of several AAV stereotypes, including without limitation, AAV-1, AAV-2, AAV-3, AAV-4, AAV-5, AAVX7, etc. Furthermore, 5′ and 3′ ITRs which flank a selected nucleotide sequence in an AAV expression vector need not necessarily be identical or derived from the same AAV stereotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable DNA molecules for use in AAV vectors will be less than about 5 kilobases (kb) in size and will include, for example, a gene that encodes a protein that is defective or missing from a recipient subject or a gene that encodes a protein having a desired biological or therapeutic effect (e.g., an antibacterial, antiviral or antitumor function). Preferred DNA molecules include those involved in dopamine metabolism, for example, AADC or TH. AAV-AADC and AAV-TH vectors have been described, for example, in Bankiewicz et al. (1997) Exper't Neurol. 144:147-156; Fan et al (1998) Human Gene Therapy 9:2527-2535 and International Publication WO 95/28493, published Oct. 26, 1995.

The selected nucleotide sequence, such as AADC or another gene of interest, is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from non-viral genes, such as the murine metallothionein gene, will also find use herein. Such promoter sequences are commercially available from, e.g., Stratagene (San Diego, Calif).

For purposes of the present invention, both heterologous promoters and other control elements, such as CNS-specific and inducible promoters, enhancers and the like, will be of particular use. Examples of heterologous promoters include the CMB promoter. Examples of CNS-specific promoters include those isolated from the genes from myelin basic protein (MBP), glial fibrillary acid protein (GFAP),and neuron specific enolase (NSE). Examples of inducible promoters include DNA responsive elements for ecdysone, tetracycline, hypoxia and aufin.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl (2), 10 mM DTT, 33 ug/ml BSA, 10 mM-50 mM NaCl, and either 40 uM ATP, 0.01-0.02 (Weiss) units T4 DNA ligase at 0[deg] C. (for “sticky end” ligation) or 1 mM ATP, 0.3-0.6 (Weiss) units T4 DNA ligase at 14[deg] C. (for “blunt end” ligation). Intermolecular “sticky end” ligations are usually performed at 30-100 [mgr]g/ml total DNA concentrations (5-100 LnM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e.g., Edge, Nature (1981) 292:756; Nambair et al. Science (1984)223:1299; Jay et al. J. Biol. Chem. (1984) 259:6311.

In order to produce rAAV virions, an AAV expression vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

For the purposes of the invention, suitable host cells for producing rAAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) are preferred in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral Ela and Elb genes (Aiello et al. (1979) Virology 94:460). The 293 cell line is readily transfected, and provides a particularly convenient platform in which to produce rAAV virions.

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication,DNA helicase activity and modulation of transcription from AAV (or other heterologous) promoters. The Rep expression products are collectively required for replicating the AAV genome. For a description of the AAV rep coding region, see, e.g., Muzyczka, N. (1992) Current Topics in Microbiol. and Immunol. 158:97-129; and Kotin, R. M. (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

The host cell (or packaging cell) must also be rendered capable of providing non AAV derived functions, or “accessory functions,” in order to produce rAAV virions. Accessory functions are non AAV derived viral and/or cellular functions upon which AAV is dependent for its replication. Thus, accessory functions include at least those non AAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

Particularly, accessory functions can be introduced into and then expressed in host cells using methods known to those of skill in the art. Commonly, accessory functions are provided by infection of the host cells with an unrelated helper virus. A number of suitable helper viruses are known, including adenoviruses; herpes viruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241-247; McPherson et al. (1985) Virology 147:217-222; Schlehofer et al. (1986) Virology 152:110-117.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCl gradients. Further, if infection is employed to express the accessory functions, residual helper virus can be inactivated, using known methods. For example, adenovirus can be inactivated by heating to temperatures of approximately 60[deg] C. for, e.g., 20 minutes or more. This treatment effectively inactivates only the helper virus since AAV is extremely heat stable while the helper adenovirus is heat labile.

Based on the rodent studies described, and on the development of MRI monitored surgical injection procedures, and results in primate studies with monkeys, including vervet monkeys, a successful strategy for the introduction, and transduction, of the hAADC vectors has been developed. Thus, a viral delivery protocol can be used for relatively short term administration, to promote long term maintenance of dopaminergic activity in otherwise-depleted striatal neurons, and, when coupled with systemic (oral where possible, IV otherwise) administration of L-dopa or L-dopa/carbidopa, offers a long term method for reducing the continuing degeneration associated with Parkinson's Disease and in particular, loss of efficacy of administration of L-dopa. MRI guided procedures for virus delivery have allowed carefully controlled selection and monitoring of injection site. Using the preferred viral vector AAV-MD-hAADC, particle titer is typically 1×10 e 12, with a range of two orders of magnitude. In any treatment, sites in both the caudate and putamen will receive injection. Each site may receive 1-2 injections, depending the severity of symptoms presented. For injections into the caudate, injection volume is 1-3 μl. Injections to the putamen are on the order of 9-11 μl. A slower infusion rate helps to ensure a larger volume distribution. Kroll et al., Neurosurgery, 38, 746-754 (1996). Accordingly, an infusion rate of approximately 0.1 μl/minute is used, in contrast to conventional rates of 1-2 μl per minute. The coordinates of the desired target within either caudate or putamen is readily determinated based on a computer algorithm. The best entry point, trajectory, and angle of entrance are planned and verified in the MRI.

Restoration, through transfection, of AADC activity to the neurons, alone, of course does not result in generation of dopamine. Systemic/preipheral administration of L-dopa, both with and without carbidopa, must be maintained at levels roughly equivalent to those administered for similar patients, according to a regimen that is quite well developed, or at the level the patient was receiving prior to experiencing a lack of effectiveness of L-dopa alone, and viral injection.

Applicants wish to stress that the method for treatment of Parkinson's Disease disclosed herein is specific and confined. Importantly, Applicants' invention focuses on a pro-drug strategy of systemic administration of L-dopa coupled with restoration of AADC activity in striatal neurons most commonly susceptible to the degenerative effects of Parkinson's Disease. Accordingly, the effective agent is the heterologous DNA carried by the viral vector, and the treatment contemplates the administration of this heterologous DNA only, and not the DNA for other enzymes implicated in the pathway, markers or the like. Similarly, the invention contemplates minimizing intrusion into and exposure of the CNS. CED Delivery Systems implicate a wide variety of neural tissues, with prolonged exposure and substantially greater opportunity for infection, toxicity and the like. Specific injection is the protocol contemplated by this invention. Coupled with systemic administration of L-dopa, transfection of the caudate and putamen neurons, where degenerative loss of AADC activity occurs in Parkinson's Disease, provides an effective, long term strategy for maintenance of Parkinson's patients.

The invention addressed herein has been described both generically, and by specific example. Except where indicated otherwise, specific examples are not intended to be limiting, and alternatives that occur to those of skill in the art, unless expressly excluded by the language of the claims set forth below are included within the scope of the invention.

1. A method of improving the effectiveness of treatment of a human patient exhibiting symptoms of Parkinson's Disease, wherein treatment is systemic administration of L-dopa, wherein said treatment is of declining effectiveness due to loss of aromatic L-amino acid decarboxylase (AADC) activity in striatal neurons of the patient's caudate and putamen regions comprising:
transfecting neurons of the caudate region, the putamen region, or both, of said human patients brain with a viral vector which comprises an expression system for a nucleic acid encoding AADC by injecting said vector into said regions, and
continuing to administer therapeutically effective amounts of L-dopa to said patient, systemically.

2. A method for treating Parkinson's Disease in a subject, said method comprising:
(a) providing a preparation comprising recombinant adeno-associated virus (rAAV) virions, wherein said virions are produced in vitro and comprise a nucleic acid sequence encoding AADC; and
(b) delivering the preparation, in vivo, to the brain of the subject, wherein said virions transduce cells and the AADC is expressed by the cells at levels that provide therapeutically effective levels of dopamine upon the oral administration of L-dopa.

3. The method of claim 1, wherein said viral vector is comprised of recombinant adeno-associated virus virions, each comprising a heterologous nucleic acid sequence encoding AADC with an appropriate promoter.

4. The method of claim 3, wherein said viral vector is directly injected into both caudate and putamen regions.

5. The method of claim 4, wherein said injection is of a sample having a viral virion concentration of 1×10e10-1×10e14 ml—and the volume per injection is from 1-3 μl for said caudate region and 9-11 μl for said putamen region.

6. The method of claim 5, wherein said injection is infused at a rate of 0.05-0.5 μl/minute.

7. The method of claim 6, wherein said infusion rate is 0.1 μl/minute, said particle titre is 1×10e12 particle/ml, and said L-dopa administration is oral or intravenous.

8. The method of claim 7, wherein said L-dopa administration is oral.
